(19) United States
(12) Patent Application Publication
(10) Pub. No.: US 2014/0128738 A1
(43) Pub. Date: May 8, 2014

(54) SYSTEM AND METHODS FOR FORMING ULTRASOUND IMAGES

(71) Applicant: FUJIFILM VisualSonics, Inc., Toronto (CA)

(72) Inventors: Christopher A. White, Toronto (CA); James Mehi, Thornhill (CA); Stanley Poon, Thornhill (CA)

(73) Assignee: FUJIFILM VISUALSONICS, INC., Toronto (CA)

(21) Appl. No.: 14/072,755

(22) Filed: Nov. 5, 2013

Related U.S. Application Data
(60) Provisional application No. 61/722,745, filed on Nov. 5, 2012.

(51) Int. Cl. A61B 8/00; A61B 8/14; A61B 5/113; A61B 8/08; A61B 5/0456

(52) U.S. Cl. CPC A61B 8/543; A61B 8/5207; A61B 5/0456; A61B 5/113; A61B 8/5276; A61B 8/145; A61B 8/4483
USPC 600/447; 600/443

(57) ABSTRACT

Systems and methods for producing three-dimensional ultrasound images are disclosed herein. In one embodiment, ultrasound image data are acquired in discrete time increments at one or more positions relative to a subject. The image data is time stamped relative to an offset to a reference point. The image data is synchronized relative to the reference point and combined to form a loop of three dimensional images.

### CROSS-REFERENCE TO RELATED APPLICATIONS

The present application claims priority to U.S. Provisional Application No. 61/722,745, titled “SYSTEMS AND METHODS FOR FORMING ULTRASOUND IMAGES,” filed on Nov. 5, 2012, which is incorporated herein by reference in its entirety.

### PATENTS AND PATENT APPLICATIONS INCORPORATED BY REFERENCE

The following patents are also incorporated herein by reference in their entireties: U.S. Pat. No. 7,052,460, titled “SYSTEM FOR PRODUCING AN ULTRASOUND IMAGE USING LINE-BASED IMAGE RECONSTRUCTION,” and filed Dec. 15, 2003; U.S. Pat. No. 7,255,648, titled “HIGH FREQUENCY, HIGH FRAME-RATE ULTRASOUND IMAGING SYSTEM,” and filed Oct. 10, 2003; and U.S. Pat. No. 7,901,358, titled “HIGH FREQUENCY ARRAY ULTRASOUND SYSTEM,” and filed Nov. 2, 2006.

### BACKGROUND

In ultrasound imaging devices, images of a subject are created by transmitting one or more acoustic pulses into the body from a transducer. Reflected echo signals that are created in response to the pulses are detected by the same or a different transducer. The echo signals cause the transducer elements to produce electronic signals that are analyzed by the ultrasound system in order to create a map of some characteristic of the echo signals such as their amplitude, power, phase or frequency shift etc. The map therefore can be displayed to a user as, for example, a 2D, 3D or 4D ultrasound image.

4D imaging involves imaging 3D data sets at different time points. Some conventional ultrasound systems utilize a 4D transducer array that can image multiple 2D slices at the same time. This is accomplished by fabricating a 2D transducer array and using sophisticated 3D beam forming hardware. While this conventional configuration is able to acquire real time 4D data sets there are a number issues: the imaging field of view tends to be small, the frame rate low, the cost is high, and the electronics required to control the transducer can be complicated and expensive. The frames rates for this configuration are low, which can be restrictive when doing cardiac imaging. This type of array has also not been created for use at high frequencies (above 20 MHz). For these reasons conventional 4D arrays are not suitable for high resolution cardiac imaging particular in small animals where the heart rate and the imaging resolution requirements can be very high. Accordingly, a need exists for acquisition of 4D data sets for cardiac imaging with a high frame rate.

### BRIEF DESCRIPTION OF THE DRAWINGS

FIG. 1 is a block diagram of an ultrasound imaging system configured in accordance with an embodiment of the disclosed technology.

FIG. 2 is a schematic view of an ultrasound imaging system in accordance with an embodiment of the disclosed technology.

FIG. 3A illustrates a graphical representation of an ultrasound image and an electrocardiogram signal in accordance with an embodiment of the disclosed technology.

FIG. 3B illustrates an ultrasound image in accordance with an embodiment of the disclosed technology.

FIG. 3C illustrates a detail view of an electrocardiogram signal in accordance with an embodiment of the disclosed technology.

FIGS. 4A and 4B illustrate examples of an ultrasound data acquisition in accordance with an embodiment of the disclosed technology.

FIG. 5 is a flowchart illustrating a process of acquiring ultrasound data and forming ultrasound images in accordance with an embodiment of the disclosed technology.

FIGS. 6A-6D are ultrasound images formed in accordance with an embodiment of the disclosed technology.

### DETAILED DESCRIPTION

The system for producing an ultrasound image using line-based image reconstruction provides an ultrasound image having an effective frame rate in excess of, for example, 500 frames per second. The system incorporates an ECG-based technique that enables significantly higher time resolution than what was previously available, thus allowing the accurate depiction of a rapidly moving structure, such as a heart, in a small animal, such as a mouse, rat, rabbit, or other small animal, using ultrasound (and ultrasound biomicroscopy). Biomicrosopy is an increasingly important application due to recent advances in biological, genetic, and biochemical techniques, which have advanced the mouse as a desirable test subject for the study of diseases, including the cardio-vascular diseases.

In one aspect, the system for producing an ultrasound image using line-based image reconstruction addresses specifically the need to image and analyze the motions of the heart of a small animal with proportionally relevant time and detail resolution. Such imaging is also applicable to imaging small structures within a human body. It also applies to other ultrasound imaging applications where effective frame rates greater than, for example, 200 frames per second are desired.

Ultrasound images are formed by the analysis and amalgamation of multiple pulse echo events. An image is formed, effectively, by scanning regions within a desired imaging area using individual pulse echo events, referred to as or ultrasound scans or lines. Each pulse echo event requires a minimum time for the acoustic energy to propagate into the subject and to return to the transducer. The image is completed by “covering” the desired image area with a sufficient number of acquisition lines, referred to as “painting in” the desired imaging area so that sufficient detail of the subject anatomy can be displayed. The number of and order in which the lines are acquired can be controlled by the ultrasound system, which also converts the raw data acquired into an image. Using a combination of hardware electronics and software instructions in a process called “scan conversion,” or image construction, the ultrasound image obtained is rendered so that a user viewing the display 50 can view the subject being imaged.

ECG signals acquired during the ultrasound scanning operation are used to time register individually the individual pulse-echo events or raw data associated with each scan line. A scan conversion mechanism utilizes the ultrasound lines, which are time registered with the ECG signal, to develop an image having an effective frame rate 60 significantly greater that the frame rate that may be obtained in real-time. A sequential series of image frames is reconstructed from the pool of time and position registered raw data to reconstruct a very high precision (i.e., high frame rate) representation of the rapidly moving structure.

### Suitable System

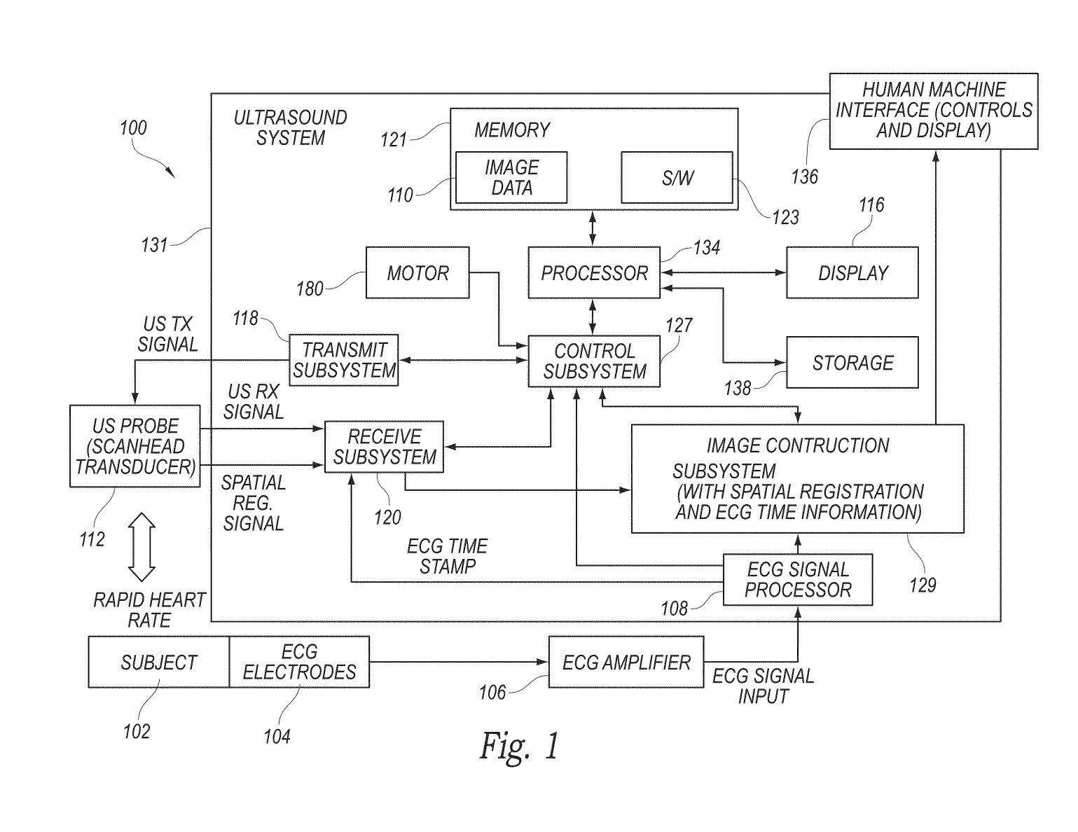

FIG. 1 is a block diagram illustrating an imaging system 100. The system 100 operates on a subject 102. An ultrasound probe 112 is placed in proximity to the subject 102 to obtain image information. The ultrasound probe generates ultrasound energy at high frequencies, such as, but not limited to, greater than 20 MHz and including 25 MHz, 30 MHz, 35 MHz, 40 MHz, 45 MHz, 50 MHz, 55 MHz, 60 MHz and higher. Further, ultrasound operating frequencies significantly greater than those mentioned above are also contemplated. The subject 102 is connected to electrocardiogram (ECG) electrodes 104 to obtain a cardiac rhythm from the subject 102. The cardiac signal from the electrodes 104 is transmitted to an ECG amplifier 106 to condition the signal for provision to an ultrasound system 131. It is recognized that a signal processor or other such device may be used instead of an ECG amplifier to condition the signal. If the cardiac signal from the electrodes 104 is suitable, then use of an amplifier 106 or signal processor could be avoided entirely.

The ultrasound system 131 includes a control subsystem 127, an image construction subsystem 129, sometimes referred to as a “scan converter”, the transmit subsystem 118, the receive subsystem 120 and the user input device 136. The processor 134 is coupled to the control subsystem 127 and the display 116 is coupled to the processor 134. A memory 121 is coupled to the processor 134. The memory 121 can be any type of computer memory, and is typically referred to as random access memory “RAM,” in which the software 123 of the invention executes. The software 123 controls the acquisition, processing and display of the ultrasound data allowing the ultrasound system 131 to display a high frame rate image so that movement of a rapidly moving structure may be imaged. The software 123 comprises one or more modules to acquire, process, and display data from the ultrasound system 131. The software comprises various modules of machine code, which coordinate the ultrasound subsystems, as will be described below. Data is acquired from the ultrasound system, processed to form complete images, and then displayed to the user on a display 116. The software 123 allows the management of multiple acquisition sessions and the saving and loading of these sessions. Post processing of the ultrasound data is also enabled through the software 123.

The system for producing an ultrasound image using line-based image reconstruction can be implemented using a combination of hardware and software. The hardware implementation of the system for producing an ultrasound image using line-based image reconstruction can include any or a combination of the following technologies, which are all well known in the art: discrete electronic components, a discrete logic circuit(s) having logic gates for implementing logic functions upon data signals, an application specific integrated circuit having appropriate logic gates, a programmable gate array(s) (PGA), a field programmable gate array (FPGA), one or more massively parallel processors, etc.

The software for the system for producing an ultrasound image using line-based image reconstruction comprises an ordered listing of executable instructions for implementing logical functions, and can be embodied in any computer readable medium for use by, or in connection with, an instruction execution system, apparatus, or device, such as a computer-based system, processor-containing system, or other system that can fetch the instructions from the instruction execution system, apparatus, or device and execute the instructions.

In the context of this document, a “computer-readable medium” can be any means that can contain, store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM), a read-only memory (ROM), an erasable programmable read-only memory (EPROM or Flash memory) (magnetic), an optical fiber (optical), and a portable compact disc read-only memory (CD-ROM) (optical). Note that the computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

The memory 121 can include the image data 110 obtained by the ultrasound system 100. A computer readable storage medium 138 is coupled to the processor for providing instructions to the processor to instruct and/or configure processor to perform steps or algorithms related to the operation of the ultrasound system 131, as further explained below. The computer readable medium can include hardware and/or software such as, by way of example only, magnetic disks, magnetic tape, optically readable medium such as CD ROM's, and semiconductor memory such as PCMCIA cards. In each case, the medium may take the form of a portable item such as a small disk, floppy diskette, cassette, or it may take the form of a relatively large or immobile item such as hard disk drive, solid state memory card, or RAM provided in the support system. It should be noted that the above listed example mediums can be used either alone or in combination.

The ultrasound system 131 can include a control subsystem 127 to direct operation of various components of the ultrasound system 131. The control subsystem 127 and related components may be provided as software for instructing a general purpose processor or as specialized electronics in a hardware implementation. The ultrasound system 131 includes an image construction subsystem 129 for converting the electrical signals generated by the received ultrasound echoes to data that can be manipulated by the processor 134 and that can be rendered into an image on the display 116. The control subsystem 127 is connected to a transmit subsystem 118 to provide an ultrasound transmit signal to the ultrasound probe 112. The ultrasound probe 112 in turn provides an ultrasound receive signal to a receive subsystem 120. The receive subsystem 120 also provides signals representative of the received signals to the image construction subsystem 129. The receive subsystem 120 is also connected to the control subsystem 127. The scan converter is directed by the control subsystem 127 to operate on the received data to render an image for display using the image data 110.

The ultrasound system 131 can include an ECG signal processor 108 configured to receive signals from the ECG amplifier 106. The ECG signal processor 108 provides various signals to the control subsystem 127. The receive subsystem 120 also receives an ECG time stamp from the ECG signal processor 108. The receive subsystem 120 is connected to the control subsystem 127 and an image construction subsystem 129. The image construction subsystem 129 is directed by the control subsystem 127.

The ultrasound system 131 can further include a motor 180 (e.g., a stepper motor, servo-torque motor, wobbler, etc.) configured to move the ultrasound probe 112. The motor 180, for example, can be configured to move the ultrasound probe 112 in one or more spatial directions (e.g., along an x, y and/or z-axis) and/or rotate the ultrasound probe 112.

The ultrasound system 131 transmits and receives ultrasound data through the ultrasound probe 112, provides an interface to a user to control the operational parameters of the imaging system 100, and processes data appropriate to formulate still and moving images that represent anatomy and/or physiology. Images are presented to the user through the interface display 116.

The human-machine interface 136 of the ultrasound system 131 takes input from the user, and translates such input to control the operation of the ultrasound probe 106. The human-machine interface 136 also presents processed images and data to the user through the display 116.

The software 123 in cooperation with the image construction subsystem 129 operate on the electrical signals developed by the receive subsystem 120 to develop a high frame-rate ultrasound image that can be used to image rapidly moving anatomy of the subject 102.

The control subsystem 127 coordinates the operation of the ultrasound probe 112, based on user selected parameters, and other system inputs. For example, the control subsystem 127 ensures that data is acquired at each spatial location, and for each time window relative to the ECG signal. Therefore, a full data set includes raw data for each time window along the ECG signal, and for each spatial portion of the image frame. It is recognized that an incomplete data set may be used with appropriate interpolation between the values in the incomplete data set being used to approximate the complete data set.

The transmit subsystem 118 generates ultrasound pulses based on user selected parameters. The ultrasound pulses are sequenced appropriately by the control subsystem 127 and are applied to the probe 112 for transmission toward the subject 102.

The receive subsystem 120 records the echo data returning from the subject 102, and processes the ultrasound echo data based on user selected parameters. The receive subsystem 120 also receives a spatial registration signal from the probe 112 and provides position and timing information related to the received data to the image construction subsystem 129.

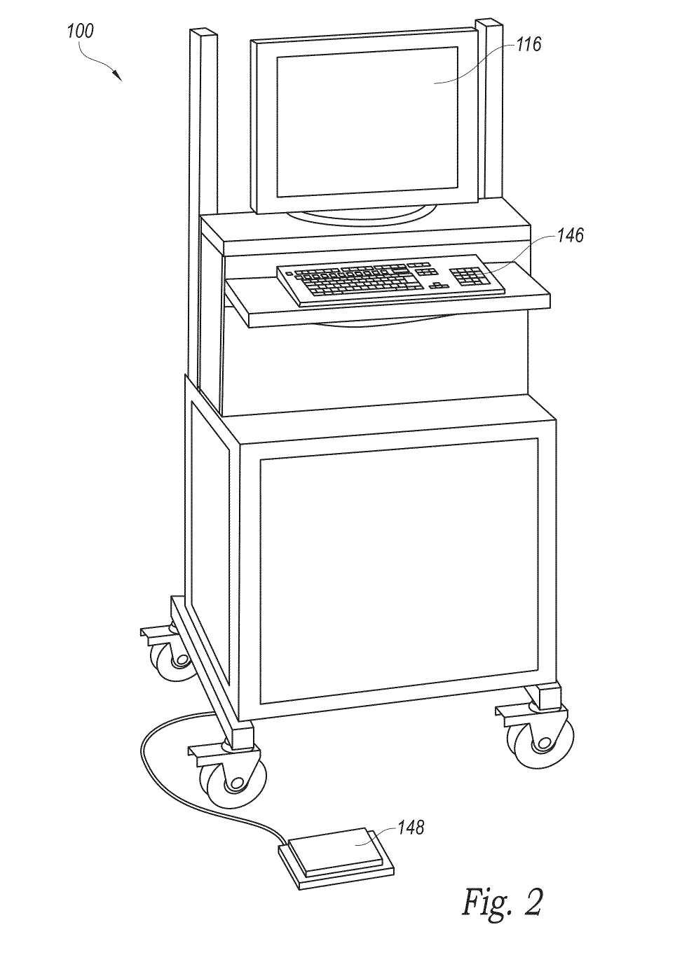

Referring to FIG. 2, an embodiment of the ultrasound system 100 is shown by way of example only. In this example, the ultrasound system 100 is a free-standing unit on casters for mobility. The human machine interface 136 includes a display 116, a keyboard 146, and a foot control 148. The control subsystem 127 and related components may be located inside a case.

### Acquisition

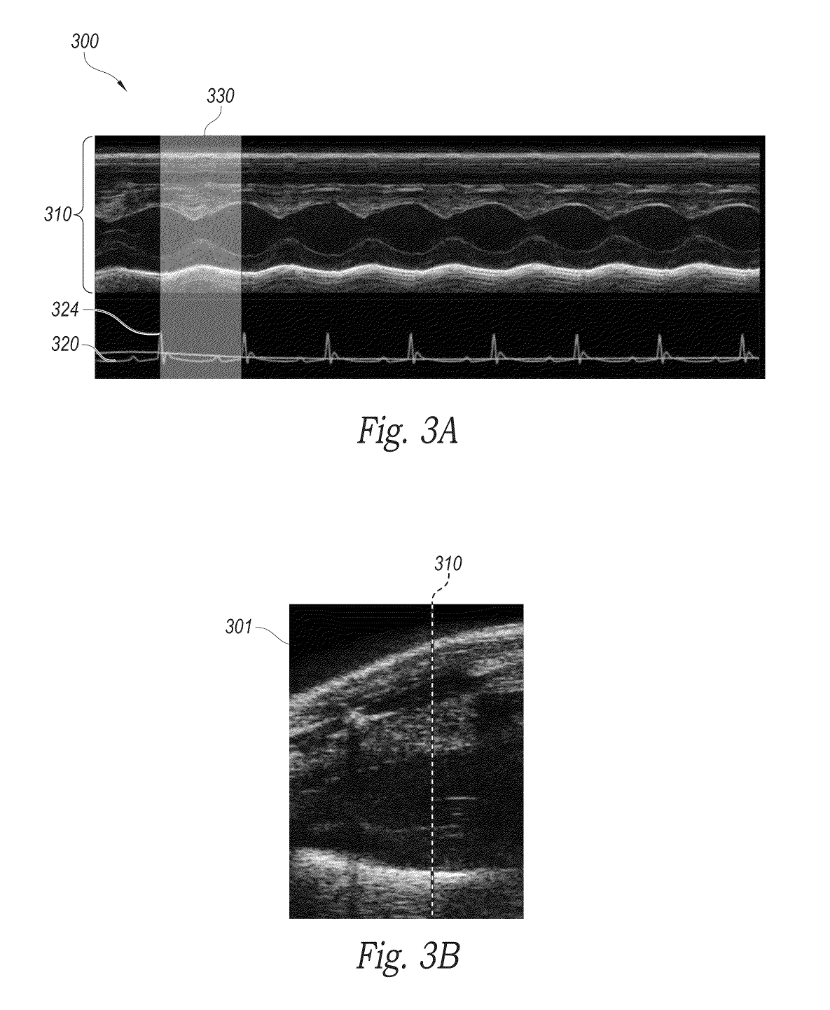

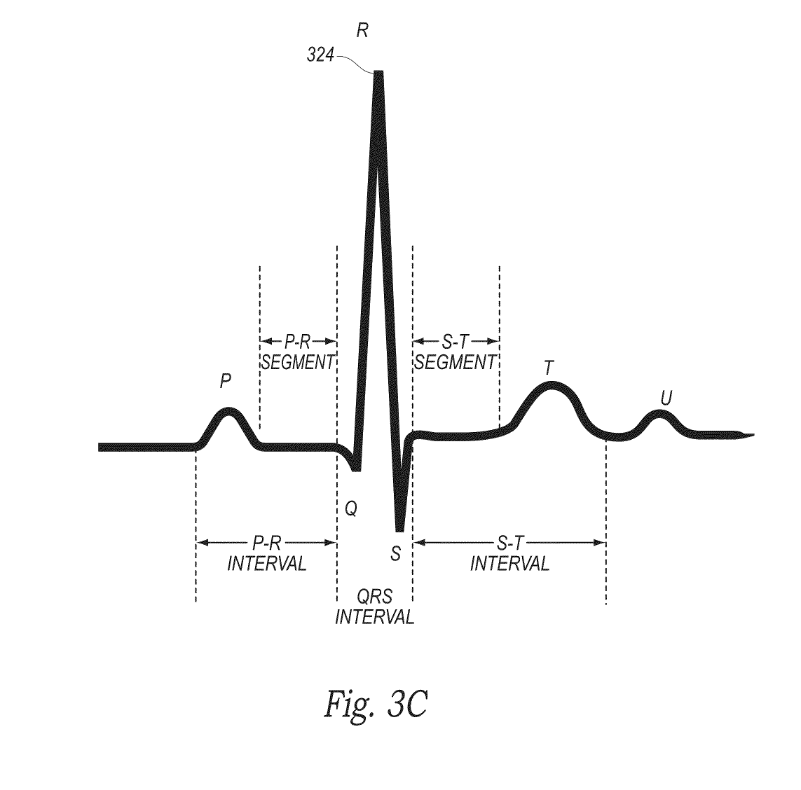

FIG. 3A shows an ultrasound image display 300 having an ultrasound trace or data set 310 and an ECG signal 320. In the illustrated embodiment, the ultrasound data set 310 is a graphical display of an M-mode ultrasound acquisition obtained from the heart of a subject (e.g., a mouse, rat, human, etc.). One complete cardiac cycle of the subject is shown by the shaded area 330 between successive r-wave events or peaks 324 (shown in detail in FIG. 3C). As those of ordinary skill will appreciate, the start of the peaks 324 generally corresponds to the diastole of a cardiac cycle. As shown in FIG. 3B, the ultrasound data set 310 is one of a predetermined number (e.g., 64, 128, 256, 512, 1024, etc.) of ultrasound data sets acquired during a measurement.

FIG. 3C illustrates a typical heart cycle with P, Q, R, T, and U features noted. While the P, Q, T, and U points are not always discernible; the R-Wave is a dominant feature and is usually pronounced and detectable. Heart compression occurs at the R-Wave (e.g., systole, when the heart contracts) and represents the point of maximum wall velocity. This is where a high temporal accuracy is preferable and, thus, the requirement for the highest frame rate. If the frame rate is too low, for example, important motion features, such as the opening and closing of the heart valve, or systolic compression may be missed. ECG triggering can be accomplished by detecting the R wave and delaying acquisition of a frame. Depending on how fast the frame acquisition is, there may be some motion drift. For example, if it takes 10 ms to acquire the frame, the heart may have moved between when the frame started and stopped acquisition. This may be seen as a skewing of the heart shape across the image from left to right. Thus both a high frame rate and a very short acquisition time can increase accuracy of the heart shape.

3D acquisitions may be acquired using a stepper motor (e.g., the motor 180) connected to a transducer (not shown). The motor can be moved through the entire range of the acquisition and at discrete points an ultrasound image is acquired. These images can be built up into a 3D volume. For cardiac imaging, unless ECG gating is employed, there may not be synchronization between these images. Each image can be acquired at a different point in the cardiac cycle thereby producing an incoherent 3D volume. Two different 3D acquisition methodologies to synchronize 3D volumes to a heart cycle are described below.

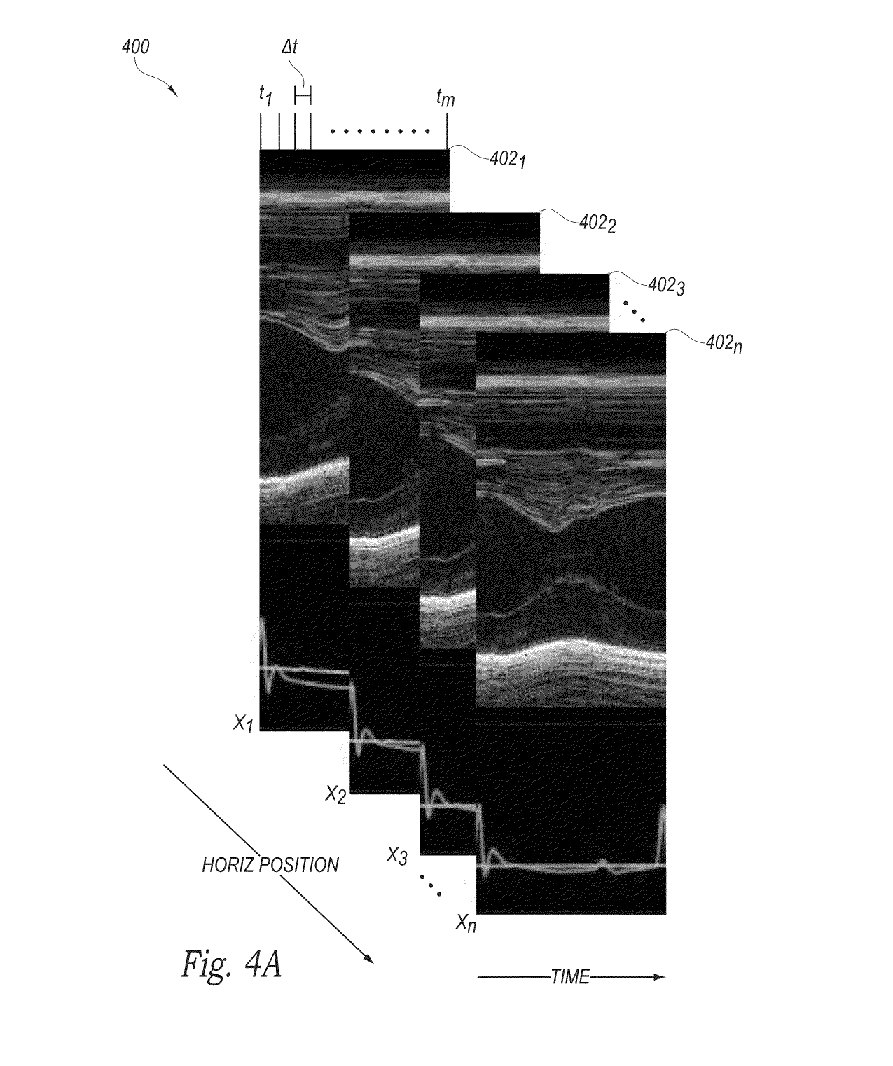

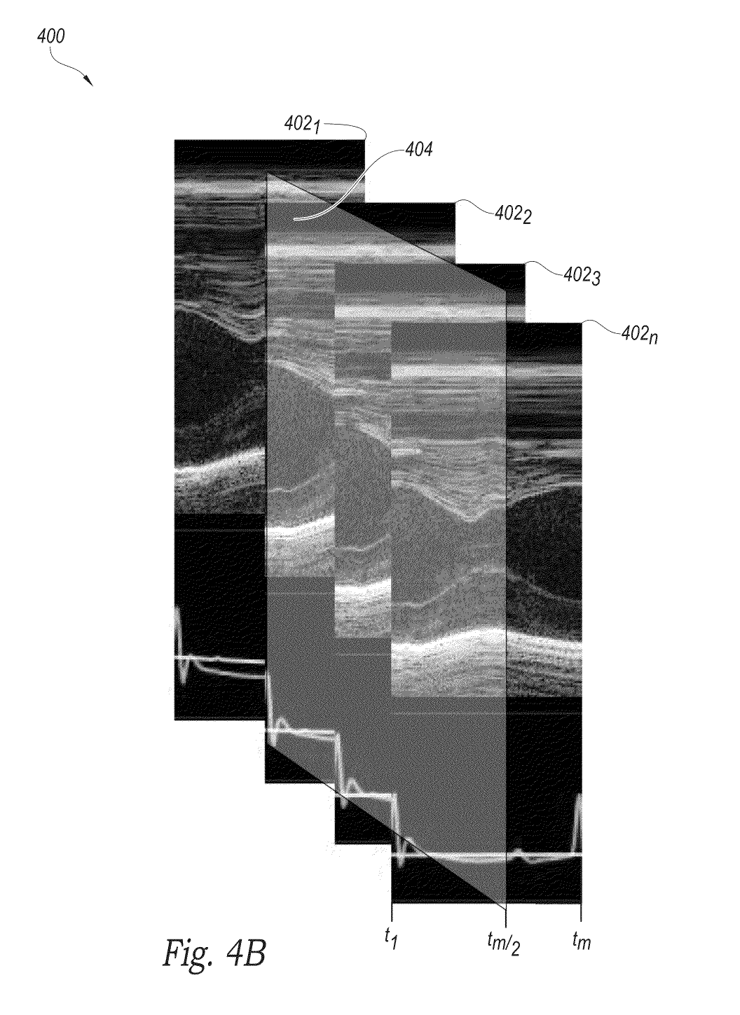

FIGS. 4A and 4B illustrate an ultrasound acquisition 400 referred to as an Electro-Kilohertz-Visualization (hereinafter referred to as “EKV”) acquisition mode. In an EKV acquisition mode, an ultrasound data line may be acquired along with, for example, an ECG, a respiration signal, and/or other physiological signals during one or more physiological cycles (e.g., a cardiac cycle, a respiration cycle, etc.) at one or more positions relative to an organ of interest in a subject (e.g., a heart). The collection of data lines is referred to as a data set and comprises of one or more data lines acquired at a single position over a time period. A stack of the one or more ultrasound data sets can be acquired in succession at different positions to form a stack of data sets as shown in FIGS. 4A and 4B. As described in further detail below, this stack of ultrasound data can be combined to form a cine loop of 2D images representing a complete cardiac cycle in one physical dimension which could be the XY plane

The use of EKV can allow an ultrasound system to retrospectively generate high frame rate ultrasound images of a complete heart cycle at any rate between (but not limited to) for example, 10 and 10,000 fps. In some embodiments, for example, each ultrasound data set can comprise one or more ultrasound data lines assigned with a unique timestamp. The timestamp can include, for example, a time offset in seconds relative to an event (e.g., a physiological event such as an r-wave peak at the beginning of a cardiac cycle). Assigning a timestamp to each of the ultrasound data lines in a data set can allow the system to synchronize ultrasound data from multiple data sets having the same relative offset from a physiological event (e.g., an r-wave peak). The assigning of timestamps further allows the system to discard data that may be associated with movement of the subject (e.g., an ultrasound data line acquired at the same time as an r-wave peak). As a result, ultrasound data lines from multiple ultrasound data sets can be synchronized relative to a regularly-repeating and/or periodic event (e.g., an r-wave peak) without having been acquired at the same exact cycle to form a cine-loop of cardiac functions through an entire heart.

In some embodiments, for example, a 5 second EKV acquisition may be sufficient to generate a cine loop at approximately 200 fps. While the frame rate is not nearly as high as the 1000 fps typically seen in some EKV images, it is enough to generate a very smooth cine loop of a complete heart cycle (of about 20 images per heart cycle). One complete EKV acquisition is then acquired for each position, for example, moved through the Z axis, of the motor 180. In this way a coherent 4D acquisition can be made in only a few minutes. To achieve a complete EKV acquisition in 5 seconds or less, for example, a linear arrayed ultrasound system may be used to allow, for example, an application of interleaved acquisitions that may effectively parallelize the data acquisition. Other data processing techniques, for example, may also be incorporated to improve performance including, for example, advanced respiration gating to remove motion artifacts, discarding of lines that do not meet selection criteria to remove motion artifacts, and/or frame rate modification to control acquisition time.

In the illustrated embodiment of FIG. 4A, a plurality of ultrasound acquisition data sets 402₁ to 402ₙ are shown that correspond to ultrasound measurements by the ultrasound system 131 at physical positions X₁ to Xₙ, respectively. At each position, for example, a predetermined number m (e.g., 1000, 2000, etc.) ultrasound data lines may be acquired and each time-stamped with a corresponding time (e.g., a first timestamp t₁, and an mth timestamp tₘ, etc.) relative to an r-wave event and separated by a predetermined discrete amount of time Δt. As explained above, by also acquiring ECG data with the timestamps, the line time ‘t’ can be correlated to the amount of time after a preceding r-wave event that the acquisition of the ultrasound line occurred.

FIG. 4B illustrates the embodiment of FIG. 4A having a shaded area 404 corresponding to an ultrasound data frame formed from the combination of synchronized ultrasound data lines from each of the data sets 402₁, 402₂, 402₃ . . . 402ₙ. In the illustrated embodiment, the shaded area 404 represents a frame of the data lines acquired at an offset time t_{m/2} (e.g., a midpoint of a cardiac cycle) relative to an r-wave event. Multiple ultrasound data frames from other times relative to an r-wave event can be joined in succession to form, for example, a cine-loop of the heart cycle.

In contrast to the 4D EKV acquisition mode described above with reference to FIG. 4A, the ultrasound system 131 can be alternatively configured to acquire, for example, a partial 4D cardiac acquisition by acquiring multiple 3D acquisitions using ECG gating. In this case each 3D acquisition would acquire only images at a specific time point in the cardiac cycle (e.g. 0 ms after the R-Wave, 10 ms after the R-Wave, 20 ms after the R-Wave, etc.). For example, in the simplest case, one would only acquire a 3D set at systole (heart contraction) and a 3D set at diastole (relaxation). The motor 180 would move through its entire range collecting images at systole, then again move through its entire range collecting images at diastole. In this way, the ultrasound system 131 can provide the cardiologist specific information at two important phases of the heart cycle from which cardiac output measurements could be made. While not a true 4D acquisition, the number of time points during the cycle can be increased to give better temporal resolution. For many calculations, a volume at systole and diastole is sufficient to provide indication of cardiac function. Higher temporal resolution can be obtained by increasing the number of 3D acquisitions acquired at different time points in the cardiac cycle. Respiration gating, for example, can be used to remove motion artifacts caused by breathing.

### 4D EKV Acquisition Example

Referring again to FIGS. 3A and 3B, as can be seen in the ultrasound data set 310, at the start of one R-Wave peak 324, the heart is in diastole; about ½ way through it meets systole; at the end of the cycle it is back in diastole at a following peak 324. The motion is cyclic and reasonably repeatable from phase to phase. Barring respiration events, general animal motion, and irregularities in heart motion, one heart cycle is fairly similar to another. Ultrasound line data that is acquired sequentially at a constant rate, for example, 1000 lines/second, 2000 lines/second, etc., is often referred to as M-Mode data or an M-Mode acquisition. A set of acquired ultrasound line data corresponding to a heart cycle can be extracted based on the position of two sequential R-Wave events. This is possible because each line of the M-Mode acquisition is time stamped, and the acquired ECG physiological signal is acquired with time stamps. The R-Wave peaks (also called events) can be detected from the ECG data and also time stamped. One process to determine which M-Mode lines are between any two R-Wave events is as follows:

```
if (M-Mode Line Time >= Previous R-Wave Time &&
  M-Mode Line Time < Next R-Wave Time)
{
  Line is between these two R-Waves
}
```

Since a whole heart cycle of data can be extracted from M-Mode, each line can also be classified according to how long after the R-Wave peak it occurred. For example, if an M-Mode acquisition rate is 1000 lines/second, each line is acquired in 1 ms. The 10th line occurs 10 ms after the R-Wave event. For a mouse with a 600 BPM heart rate, for example, an entire heart cycle is 100 ms long. Therefore, 100 acquisition lines are needed to complete the cycle. Each line captures the heart in a different phase of its motion.

If after each M-Mode acquisition the horizontal position of the acquisition is changed (for example 0 mm, then shift left 0.1 mm, and so on), this process can be repeated for enough positions to cover the entire heart area (e.g., 100 to 200 times to complete the entire image). As noted above, 1 cardiac cycle of data can be extracted from each acquisition and organized similarly to the preceding data set. By arranging the image data in this manner, for example, a 3D block of data can be built up. In one dimension is a horizontal position across the heart, and in another dimension we have time across the heart cycle (FIG. 4A).

In this way, a complete image of the heart can be extracted for any time point. Combining the data lines at a point ½ way through the heart cycle, for example, for each acquisition position can generate an image of the heart at systole. As discussed above with reference to FIG. 4, an entire cine-loop of data can be created by repeating this process for each time point in the acquisition at uniform time points (for example 0 ms, 1 ms, 2 ms after the R-Wave time). If the acquisition rate is 1000 Hz, for example, the resulting image can be formed into a cine loop of the complete heart cycle at 1000 Hz by extracting data at 1 ms intervals. This is the basic process of generating an EKV cine loop.

To perform the method above, several factors can be measured and accounted for to improve accuracy including, for example, irregular heart motion respiration, data management, image formatting, total acquisition time, etc.

In a first example acquisition, 100 frames of a heart cycle may be acquired in 100 ms at 1000 Hz with the following parameters:

Image size=256 data sets

Time per data set=100 ms

Total Time=26 seconds

However, as those of ordinary skill in the art would appreciate, it can be difficult to trigger directly on an R-Wave event. More data may be collected to ensure that the entire cardiac cycle is acquired and the unneeded data can be trimmed from the acquisition.

Accordingly in a second example acquisition, the frames may be acquired with the following set of parameters:

Image size=256 data sets

Time per data set=200 ms

Total Time=51 seconds

In the second example acquisition, the image may be formed correctly. However, in some instances, reconstruction of the image may result in one or more disjointed lines. For example, some lines may move together, while other lines may be out of phase. This may be the result of, for example, heart motion irregularities and/or respiration motion. For example, some of lines in the data sets may be acquired during a respiration cycle and not necessarily in phase with lines from neighboring data sets. In some other instances, for example, lines that are not acquired during respiration may still be out of phase with neighboring lines each heart cycle is not necessarily exactly the same.

To account for these irregularities, a third example acquisition may be performed. As those of ordinary skill of the art would appreciate, respiration occurs on a cycle of time (e.g., 2 to 3 seconds). To acquire a data set with at least some data lines not in a respiration region, data may be acquired for a period corresponding to the length of the respiration cycle with the following parameters:

Image size=256 data sets

Time per data set=2000-3000 ms

Total Time=512 to 768 seconds

In the third example with the parameters above, the acquisition time may take 512-768 seconds (approximately 8.5-12.8 minutes). In the case of imaging in small animals, a total acquisition time of ten minutes may be difficult to perform for at least the reason that it may not feasible to keep a subject (e.g., a small animal) still for such a period of time.

The systems and methods described herein provide the advantage of acquiring lines in several data sets (e.g., 25) simultaneously or almost simultaneously such that each data set in the examples above does not have to be imaged individually. Techniques such as respiration gating, line exclusion, line decimation, line interpolation, and/or focused averaging, or multi-line acquisition in which two or more lines are acquired simultaneously through parallel beamforming can also be implemented to increase effective frame rate. As those of ordinary skill in the art would appreciate, this is possible because more than one ultrasound data line can be acquired “at the same time”. If the beamformer architecture supports it, two or more ultrasound lines corresponding to two or more different horizontal positions can be processed simultaneously from the same set of acquired data. The total acquisition time would decrease by a factor equal to the number of parallel lines processed by the beamformer.

By using linear array techniques such as, for example, interleaving multiple lines from different data sets at the same time, the acquisition time can be decreased substantially. In the third example above, acquiring 25 lines simultaneously (rather than one at a time), the acquisition time can be reduced to approximately 20 seconds (256 lines/25 lines per second). For example, a single ultrasound data line from a specific horizontal position may only take 10 micro seconds (μs) to acquire (i.e., a line acquisition time period of 10 μs). The 10 μs frame acquisition time period used as an example here is determined by the two-way time of flight of ultrasound to the deepest point in the image. If the desired frame rate is 1000 Hz, then 990 μs will elapse before another ultrasound data line needs to be acquired from the same horizontal position. A total of 1 ms will elapse between successive ultrasound lines acquired form the same horizontal position. During the remaining 990 μs time interval, ultrasound data from other horizontal positions may be acquired, each of which takes 10 μs. Each ultrasound data line is time stamped with information indicating the time delay relative to the relevant ECG feature. In this case, data sets from 100 different horizontal positions could theoretically be acquired during the 1 msec time interval, if the hardware supports this feature.

### Processing

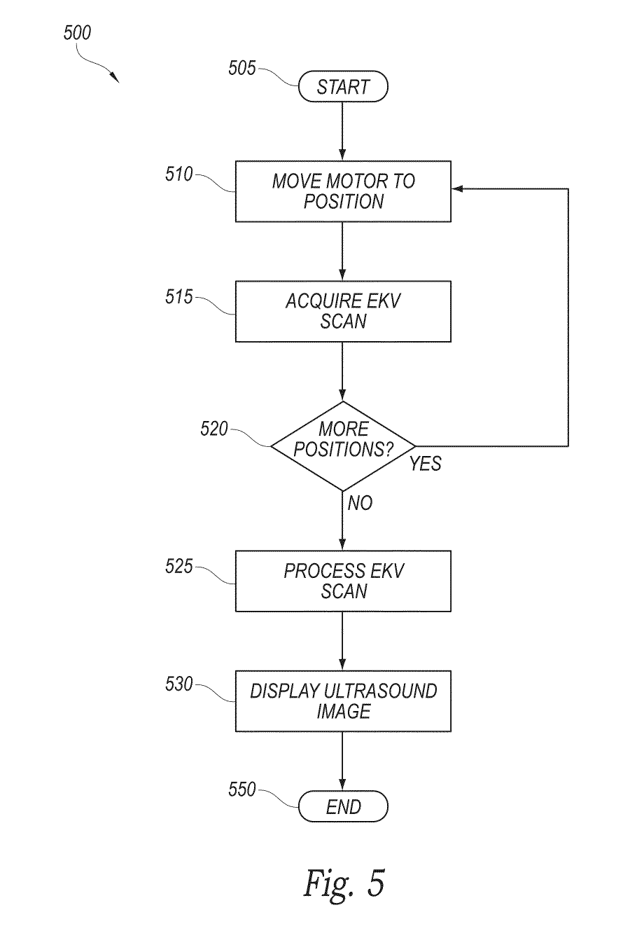

FIG. 5 is a flowchart illustrating the operation of a system 500 for producing an EKV four-dimensional ultrasound image. The blocks in the flowcharts may be executed in the order shown, out of the order shown, or concurrently.

At block 505, the system 500 is initialized. At block 510, a motor (e.g., the motor 180) configured to control the movement of an ultrasound probe (e.g., the ultrasound probe 112) is moved into a first position. The ultrasound probe can include, for example, a linear transducer array of one or more elements and be made from, for example, PZT, CMUTs, PMUTs, and/or any other suitable material known in the art.

At block 515, a complete EKV ultrasound data is acquired at the first position. The system can, for example, acquire one ultrasound line (e.g., an m-mode line) at a time or, as described above, acquire a plurality of ultrasound lines simultaneously, thereby reducing the acquisition time for an EKV data set at each position. As described above, several different parameters can be selected by the system 500 during acquisition based on, for example, a heartbeat of the subject, a desired resolution, a desired frame rate, and/or a desired acquisition time. Other parameters than the parameters listed above may also be input and/or determined by the system 500 during acquisition. As discussed above with reference to FIGS. 4A and 4B, data for each frame at each line can be associated with a time stamp that can be used to synchronize frames during processing.

At block 520, the system 500 determines whether additional positions remain to be imaged. If so, the system 500 returns to block 510 wherein the motor is moved to a next position and the system repeats the acquisition of data at block 515 at the next position. The number of positions would be dependent upon the size of the object being imaged and the desired spatial resolution required, for example, 0.5 mm spacing.

At block 525, the individual EKV data sets are processed, synchronized and combined to form a plurality of ultrasound image frames. During processing, the EKV data can be synchronized by detecting, for example, at least a first R-wave peak and a second R-wave peak for each line acquisition. Data acquired during the period between the first and second R-wave peaks can be, for example, saved by the system 500 while the data acquired outside of this period can be discarded by the system 500. As described above techniques such as, for example, ECG and/or respiration gating may also be implemented in the data acquisition and/or processing.

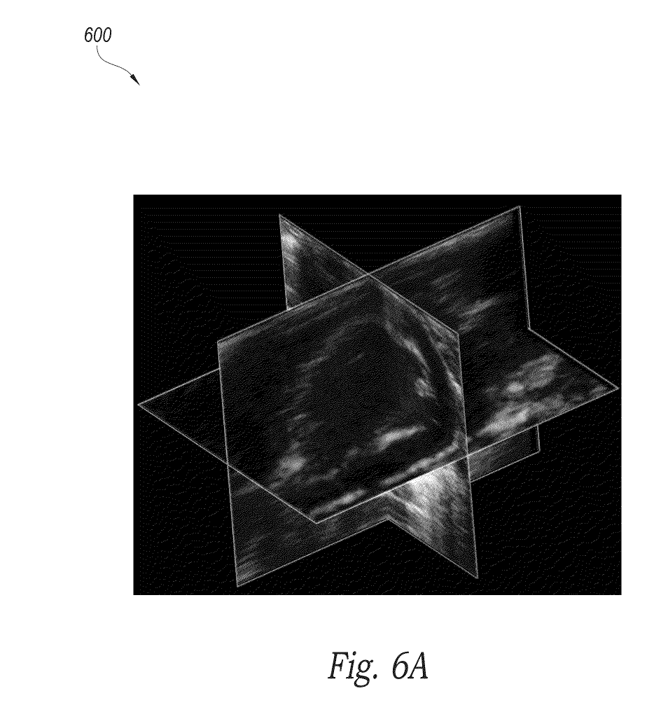

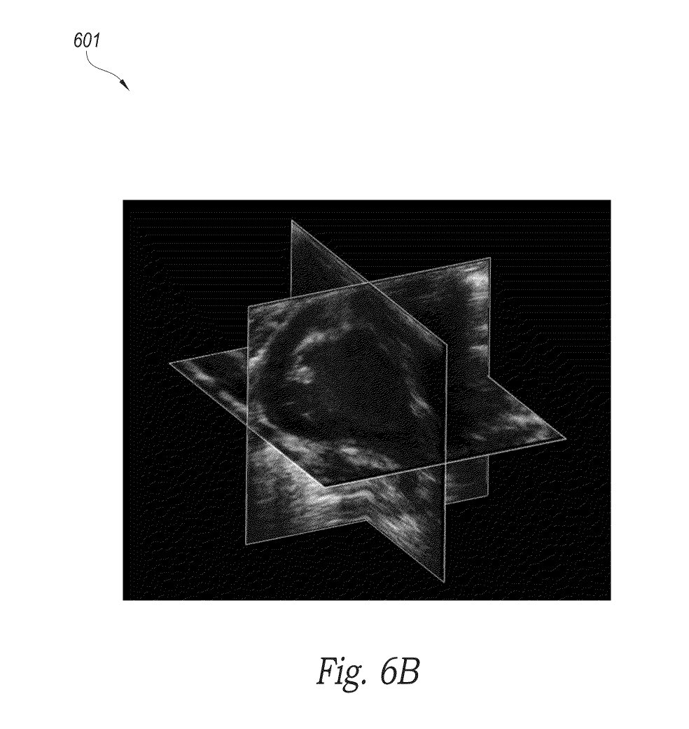

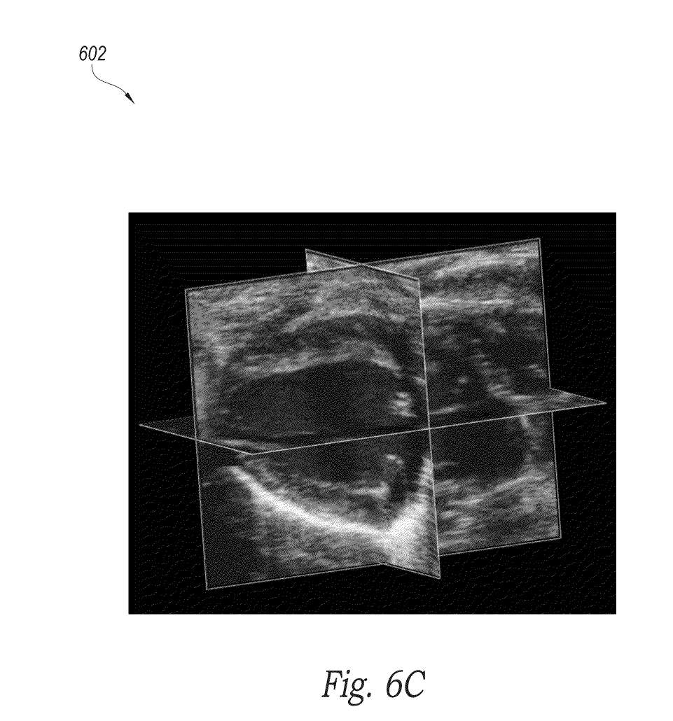

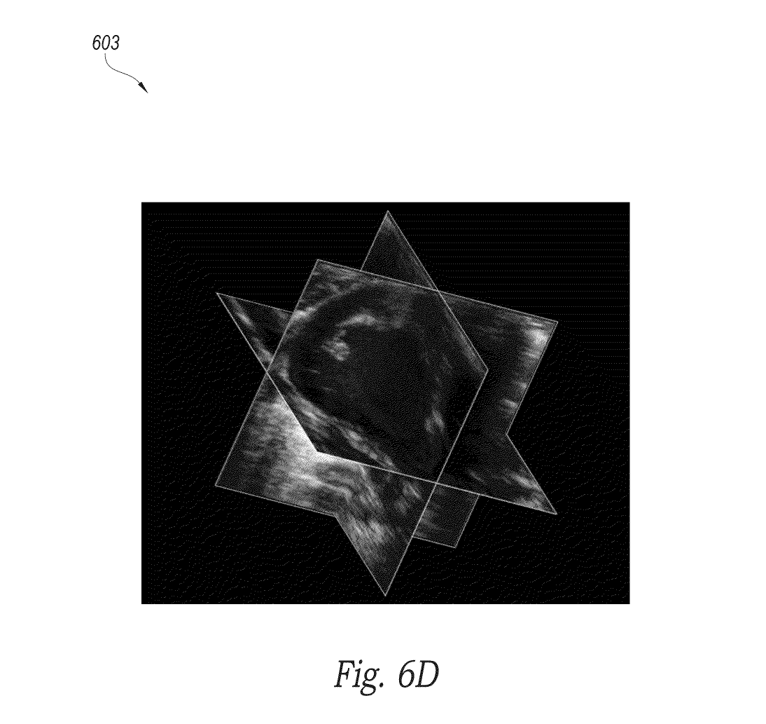

At block 530 the resulting image frames can be used to form and display, for example, 2D, 3D, and or 4D ultrasound images. The display frame rate may be the same as the “real-time” acquisition frame rate, or may be slowed down in order to allow the visualization of fast moving structures within the image. The rate at which the displayed image is played back may be user adjustable control. The images can be selectively presented to the user. For example, the user may be presented with a series of 2D or 3D images that the user can page through or browse. In addition the user may choose to view the data as a 4D cineloop (e.g., a video image of 3D images). FIGS. 6A-6D depict four examples of ultrasound 3D images produced by the techniques disclosed herein.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the disclosed technology is not intended to be exhaustive or to limit the disclosed technology to the precise form disclosed above. While specific examples for the disclosed technology are described above for illustrative purposes, various equivalent modifications are possible within the scope of the disclosed technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

The teachings of the disclosed technology provided herein can be applied to other systems, not necessarily the system described above. The elements and acts of the various examples described above can be combined to provide further implementations of the disclosed technology. Some alternative implementations of the disclosed technology may include not only additional elements to those implementations noted above, but also may include fewer elements.

These and other changes can be made to the disclosed technology in light of the above Detailed Description. While the above description describes certain examples of the disclosed technology, and describes the best mode contemplated, no matter how detailed the above appears in text, the disclosed technology can be practiced in many ways. Details of the system may vary considerably in its specific implementation, while still being encompassed by the disclosed technology disclosed herein. As noted above, particular terminology used when describing certain features or aspects of the disclosed technology should not be taken to imply that the terminology is being redefined herein to be restricted to any specific characteristics, features, or aspects of the disclosed technology with which that terminology is associated. In general, the terms used in the following claims should not be construed to limit the disclosed technology to the specific examples disclosed in the specification, unless the above Detailed Description section explicitly defines such terms.

I/We claim:

1. An ultrasound imaging system, comprising:
an ultrasound transducer;
a transmit subsystem configured to generate ultrasound energy at frequencies of 20 MHz or greater;
a receive subsystem configured to receive ultrasound echo data during a plurality of recurring physiological events, wherein the ultrasound echo data includes ultrasound data lines received from a first position and a second position;
a detector configured to indicate the occurrence of the recurring physiological events in the subject; and
a processor configured to execute one or more instructions, wherein the instructions include instructions for—
combining ultrasound data lines received from the first position and received from the second position that were acquired at substantially the same time after an occurrence of the physiological event to produce three-dimensional image data representing movement of the subject; and
forming, from the combined data, a synchronized loop of three-dimensional image frames showing movement of the subject.

2. The system of claim 1 wherein the detector is a signal processor configured to receive electrocardiogram (ECG) signals from the subject, wherein the signal processor is configured to analyze the ECG signals to detect at least a first peak, and wherein the first peak corresponds to the physiological event.

3. The system of claim 2 wherein the first peak corresponds to an R-wave.

4. The system of claim 1 wherein the instructions include instructions for—
detecting a movement of the subject associated with respiration; and
discarding ultrasound echo data received during the respiration movement.

5. The system of claim 1, further comprising a motor configured to move the transducer from the first position to at least the second position.

6. The system of claim 1 wherein the ultrasound transducer is an arrayed transducer.

7. The system of claim 6 wherein the three-dimensional image frames comprise ultrasound data lines from different positions in the subject, wherein ultrasound data lines from a position in the subject are received at a first time and a second time, and wherein ultrasound data lines from additional positions in the subject are received during the interval between the first time and the second time.

8. The system of claim 7 wherein the synchronized loop is formed from ultrasound echo data received during a time period less than or equal to twenty seconds.

9. The system of claim 8 wherein the synchronized loop comprises a frame rate of at least 200 frames per second, and a linear resolution greater than or equal to 256 lines.

10. The system of claim 7 wherein the instructions include instructions for—
detecting a point having a first depth in at least one of the three-dimensional image frames; and
determining the number of ultrasound data lines that can be acquired during the interval based on the first depth of the detected point.

11. A method of operating an ultrasound system to produce an ultrasound image, comprising:
generating ultrasound energy;
transmitting the ultrasound energy from a transducer into a subject;
receiving ultrasound echo data from the subject during an acquisition period,
wherein the ultrasound echo data includes echo data acquired along a plurality of data lines, and wherein the generating, transmitting, and receiving are performed at a first position to form a first data set of first data lines, and performed at a second position to form a second data set of second data lines;
processing the first data set and the second data set to combine data lines received at substantially the same time after an occurrence of a recurring physiological event to produce three-dimensional image data representing movement of the subject; and
forming, from the processed data, a synchronized loop of three dimensional image frames showing movement of the subject.

12. The method of claim 11 wherein the generating comprises generating ultrasound energy at a frequency greater than or equal to about 20 MHz.

13. The method of claim 11, further comprising:
acquiring electrocardiogram (ECG) signals from a heart of the subject; and
detecting at least a first peak in a waveform of the ECG signals, wherein the first peak corresponds to the recurring physiological event.

14. The method of claim 11, further comprising:
detecting a respiration movement of the subject; and
discarding ultrasound echo data received during the respiration movement.

15. The method of claim 11 wherein transmitting the ultrasound energy comprises transmitting ultrasound energy from a linear array ultrasound transducer.

16. The method of claim 15 wherein receiving ultrasound echo data from the subject comprises—
receiving ultrasound data lines from a position in the subject at a first time and a second time: and
receiving ultrasound data lines from additional positions in the subject during the interval between the first time and the second time.

17. The method of claim 16, further comprising:
detecting a point having a first depth in at least one of the three-dimensional image frames; and
determining the number of additional positions from which ultrasound data lines can be acquired during the interval based on the first depth of the detected point.

18. The method of claim 11 wherein receiving ultrasound echo data comprises receiving ultrasound echo data during an acquisition period less than or equal to five seconds.

19. The method of claim 18 wherein the synchronized loop comprises a frame rate of at least 200 frames per second, and a linear resolution greater than or equal to 256 lines.
